(19) United States
(12) Patent Application Publication
(10) Pub. No.: US 2025/0147020 A1
(43) Pub. Date: May 8, 2025

(54) Method and devices to increase the sensitivity of lateral flow assays

(71) Applicant: ABC Biotechnology Inc., Wynnewood, PA (US)

(72) Inventor: Xiaofeng Xia, Wynnewood, PA (US)

(21) Appl. No.: 18/502,189

(22) Filed: Nov. 6, 2023

(51) Int. Cl. G01N 33/543; G01N 33/94

(52) U.S. Cl. CPC G01N 33/54388; G01N 33/9486

(57) ABSTRACT

The present invention relates to a novel lateral flow assay method and devices using a container separate from the strip to contain the dried nanoparticle conjugate. The method and devices can significantly improve the sensitivity of the lateral flow assay. It can be used in both sandwich and competitive immunoassays or non-immunological assays in the lateral flow format. In specific embodiments, the methods and devices are advantageous for identifying fentanyl and analogs in a sample.

### BACKGROUND OF THE INVENTION

Lateral flow assays are widely used for the rapid detection of analytes. Many lateral flow assay devices and methods are known in the art. Typically, a lateral flow assay device is constructed as a test strip consisting of a sample pad, a conjugate pad, an analytical membrane, and an absorbent pad. The sample pad serves as the first contact with the sample, and it can also provide sample pretreatment functions including filtration, pH adjustment, ionic strength adjustment, detergent treatment, etc. The conjugation pad is where gold conjugates, such as gold conjugated antibodies, are dried and stored. The conjugation pad is also where the analytes first contact the bioreceptor and complete the binding reaction. The analytical membrane is typically made of nitrocellulose, although other materials are also used. The analytical membrane is often spotted with one test line and one control line, where the nanoparticle conjugates are retained to show detectable lines. The absorbent pad maintains the capillary flow through the membrane and is used to absorb all the reagents that are not taken up by the test and control lines.

Lateral flow assays are commonly carried out in two different formats: sandwich format and competitive format. The sandwich format is usually used to detect relatively large analytes such as proteins. It requires two different bioreceptors such as antibodies to bind at least two distinct epitopes. One of the bioreceptors is usually deposited on the analytical membrane to form a test line, while the other bioreceptor is conjugated to nanoparticles, such as gold nanoparticles, and used for detection. Analyte binding to both bioreceptors will form a sandwich structure and be detected at the test line position. In contrast, the competitive format is used to detect small analytes, such as chemical compounds including drugs of abuse. Competitive lateral flow assay can be carried out by spotting the test line with the analyte molecule (usually in protein conjugated format) and using a nanoparticle-conjugated analyte specific bioreceptor for detection. In the absence of analyte, the nanoparticle-conjugated bioreceptor will be retained on the test line and be detected. In the presence of analyte, it will compete with the analyte on the test line to bind to the bioreceptor, leading to the reduction or disappearance of signal on the test line. Alternatively, the competitive lateral flow assay can be carried out by spotting the test line with the analyte specific bioreceptor and using the nanoparticle-conjugated analyte for detection.

There has been a continuous increase in the number of overdose deaths in the United States, with the majority cases caused by fentanyl and its analogues, collectively termed fentanyls. The key feature of fentanyls is their high potency. Fentanyl is approximately 100 times more potent than morphine, and the fentanyl analog carfentanil is reported to be 10,000 times more potent than morphine. Lateral flow assays for detecting the presence of fentanyls have been developed and commercialized as test strips. All of these strips use the competitive lateral flow assay format and have a reported sensitivity of 100 ng/ml (or higher concentration) for fentanyl and other fentanyl analogs. Considering the high potency of fentanyls, improving the sensitivity of the lateral flow assays are highly demanded. The present invention serves this unmet need.

### SUMMARY OF THE INVENTION

In one aspect, the present invention relates to a method for detecting an analyte in a sample, comprising the steps of:
a) applying the sample to a container, wherein the container contains dried nanoparticle conjugates;
b) rehydrating the dried nanoparticle conjugates in the container, such that the analyte in the sample is in contact with the nanoparticle conjugate;
c) applying the sample to the sample zone of a lateral flow chromatographic test strip;
d) detecting the analyte on the detection zone of a lateral flow chromatographic test strip.

In one embodiment, the nanoparticle is selected from the group consisting of gold nanoparticles, silver nanoparticles, platinum nanoparticles, carbon nanoparticles, latex beads, magnetic nanoparticles, quantum dots, upconverting nanoparticles, and liposomes.

In one embodiment, the container is made of one or more materials selected from the group consisting of plastic, paper, metal, glass, and rubber.

In one embodiment, the method further comprises the step of collecting the sample on a swab and then dissolving the sample in a solution.

In one embodiment, the lateral flow chromatographic test strip is put in the sample container and used as a dipstick.

In one embodiment, the sample is transferred from the container to the lateral flow chromatographic test strip using a transfer pipet. In one embodiment, the lateral flow chromatographic test strip is in a cassette.

In one embodiment, the detection of the analyte is achieved by an immunoassay. In one embodiment, the immunoassay uses an antibody selected from polyclonal antibodies, monoclonal antibodies, antibody fragments or nanobodies.

In one embodiment, the detection of the analyte is achieved by a non-immunological assay. In one embodiment, the detection of the analyte uses analyte binding reagents selected from aptamers, DNAs, RNAs, proteins, polymers, or small molecule compounds.

In one embodiment, the analyte is an antigen from a pathogen.

In one embodiment, the analyte is a disease marker.

In one embodiment, the analyte is a low molecular weight compound, including drugs of abuse.

In one embodiment, the analyte is a DNA molecule.

In one embodiment, the analyte is an RNA molecule.

In one embodiment, the lateral flow assay is carried out in sandwich immunoassay format.

In one embodiment, the lateral flow assay is carried out in competitive immunoassay format. In one embodiment, the lateral flow assay is carried out in competitive immunoassay format, wherein the nanoparticle is conjugated to the analyte binding molecule. In one embodiment, the nanoparticle is conjugated to the analyte. In one embodiment, the nanoparticle is conjugated to a conjugated analyte.

In one embodiment, the lateral flow assay result is read by naked-eye detection.

In one embodiment, the lateral flow assay result is read by a strip reader. In one embodiment, the lateral flow assay result is read by optical methods selected from absorbance reading, intensity reading, fluorescent reading, light spectrum reading, or luminescent reading. In one embodiment, the lateral flow assay result is read by non-optical methods selected from magnetic reading, thermal reading, or electrochemical reading.

In one aspect, the present invention relates to a kit for identifying fentanyl and fentanyl analogs in a sample using a method comprising the steps of:
e) applying the sample to a container, wherein the container contains dried nanoparticle conjugates;
f) rehydrating the dried nanoparticle conjugates in the container, such that the analyte in the sample get full contact with the nanoparticle conjugates;
g) transferring the above samples to apply to the sample zone of a lateral flow chromatographic test strip;
h) detecting the analyte on a detection zone of the lateral flow chromatographic test strip.

In one aspect, the present invention relates to a device for detecting an analyte in a sample, comprising:
a) at least one container with dried nanoparticle conjugate, and/or
b) at least one lateral flow test strip, wherein the lateral flow matrix comprises at least one sample zone and at least one detection zone.

In one aspect, the present invention relates to a lateral flow cassette kit for identifying fentanyl and analogs in a sample using a device for detecting an analyte in a sample, comprising: at least one container with dried nanoparticle conjugates, and/or at least one lateral flow test strip, wherein the lateral flow matrix comprises at least one sample zone and at least one detection zone,

further comprising at least one container with dried antibody nanoparticle conjugate, and at least one lateral flow test strip cassette.

In one embodiment, said sample is a fluid selected from blood, plasma, serum, urine, eye fluid, sweat, CSF, saliva or other body fluid.

In one embodiment, said sample is a solution or buffer, with or without the molecule to be detected.

In one embodiment, said sample is a swab sample.

In one aspect, the present invention relates to a lateral flow dipstick kit for identifying fentanyl and analogs in a sample using a device for detecting an analyte in a sample, comprising: at least one container with dried nanoparticle conjugate, and/or

at least one lateral flow test strip, wherein the lateral flow matrix comprises at least one sample zone and at least one detection zone, further comprising at least one container with dried antibody nanoparticle conjugates, and at least one lateral flow test strip dipstick.

In one embodiment, said sample is a fluid selected from blood, plasma, serum, urine, eye fluid, sweat, CSF, saliva or other body fluid.

In one embodiment, said sample is a solution or buffer, with or without the molecule to be detected.

In one embodiment, said sample is a swab sample.

In one aspect, the present invention relates to a workflow of a kit for identifying fentanyl and analogs in a sample using a device for detecting an analyte in a sample, comprising: at least one container with dried nanoparticle conjugate, and/or

at least one lateral flow test strip, wherein the lateral flow matrix comprises at least one sample zone and at least one detection zone, further comprising at least one container with dried antibody nanoparticle conjugate, and at least one lateral flow test strip dipstick, which involves applying liquid samples to dissolve the dried gold nanoparticle in the tube, rehydrating the gold nanoparticles by shaking the tube, and then applying the test strip.

In one aspect, the present invention relates to a method for reading lateral flow assay results as illustrated in FIG. 13, by placing the test strip on the side of a drawing, to compare with the negative and positive readouts.

### BRIEF DESCRIPTION OF THE DRAWINGS

The following detailed description of preferred embodiments of the invention will be better understood when read in conjunction with the appended drawings. For the purpose of illustrating the invention, these are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts a schematic view of one embodiment of a device according to the present invention (FIG. 1A) compared to a conventional lateral flow device (FIG. 1B). The present invention includes a separate container, e.g. a vial, in which the dried nanoparticle conjugates are stored and dried.

FIG. 2 depicts an example of the device according to the present invention (Left), compared to a conventional lateral flow strip (Right).

FIG. 3 depicts an example of drying nanoparticle in a tube or vial.

FIG. 4 depicts an example of drying nanoparticle in plastic wells.

FIG. 5 depicts a schematic view of the present invention carried out in sandwich lateral flow immunoassay format.

FIG. 6 depicts a schematic view of the present invention carried out in competitive lateral flow immunoassay format, using nanoparticle conjugated antibodies.

FIG. 7 depicts a schematic view of the present invention carried out in competitive lateral flow immunoassay format, using nanoparticle conjugated antigens.

FIG. 8 depicts improved sensitivity of a competitive lateral flow assay detecting fentanyl. Left is the present invention using the lateral flow strip as a dipstick on a sample containing 5 ng/ml fentanyl. Right is using a conventional test strip on a sample containing 50 ng/ml fentanyl.

FIG. 9 depicts an example of the device comprising a sample vial, a transfer pipet, and a lateral flow test strip.

FIG. 10 depicts an example of the device comprising a sample vial, a transfer pipet, and a lateral flow test strip sealed in a plastic cassette.

FIG. 11 depicts the use of the device with a plastic cassette for detecting fentanyl in a urine sample.

FIG. 12 depicts the sensitivity of the cassette device of the present invention for detecting various fentanyl analogs.

FIG. 13 depicts the instruction of using a dipstick device for detecting fentanyl in urine samples. The steps comprise: 1) collect urine: collect urine in the provided urine cup; 2) add 6 drops: pull off the test tube cap, use the dropper to add 6 drops of urine to the tube bottom, discard the dropper; 3) shake 20 seconds: gently shake the tube 20 seconds until the red pellet at the tube bottom disappears and the sample turns pink; 4) put strip in tube: place the tube in the tube stand in the box, take the strip out of the pouch, put the strip into the tube, arrows facing down; 5) wait 5 minutes: cap the tube and wait 5 minutes.

FIG. 14 depicts the sensitivity of the dipstick device of the present inventions for detecting fentanyl in human urine samples.

### DETAILED DESCRIPTION

The present invention includes methods and devices to improve the sensitivity of lateral flow assays, while keeping the method low-cost, convenient, and fast. The invention comprises a container (such as a tube or vial) that contains dried nanoparticle conjugates, in addition to a lateral flow test strip that includes at least one immobilized capturing reagent. In one embodiment, the sample is first added to the container to re-hydrate and interact with the nanoparticle conjugate before it is applied to the lateral flow test strip to complete the assay. In preferred embodiments, the method and device use a competitive lateral flow assay format. In other preferred embodiments, the method and device use a sandwich lateral flow assay format.

The devices and methods of the present invention improve the sensitivity of lateral flow assays. In the devices and methods of the present invention, the sample is fully contacted with the nanoparticle conjugates in the separate container before it is applied to the test strip, increasing the effective analyte concentration to detect its presence in lower amounts. The device of the present invention has no liquid component which is advantageous for production, storage and transportation. In a specific embodiment, the present method and device are used for detecting fentanyl and analogs in samples. The present invention improves sensitivity by at least 10 times compared to regular lateral flow method. These advantages will be fully described in view of the below.

The present invention relates to a lateral flow method and devices for detecting an analyte in a sample, wherein the sample to be analyzed is first applied to a sample container before it is applied to a chromatographic carrier such as a lateral flow strip. The sample container is made by drying nanoparticle conjugate in it. Re-hydration of the dried nanoparticle conjugate with the sample ensures full contact with the analyte which may be present in the sample, increasing detection sensitivity. The analyte of interest is detected on the carrier by means of a binding assay such as an immunoassay.

The use of a separate container with dried nanoparticle distinguishes the present invention from prior art. In some embodiments, the container of the present invention can be made from a material selected from plastic, metal, paper, glass, rubber, or a mixture of these materials.

In some embodiments, the chromatographic carrier used in the present invention is a lateral flow test strip, comprising a sample zone, an analytic zone, and an absorbent zone. A conjugation zone is not necessary for the present invention.

In a specific embodiment, the device of the present invention comprises a tube or vial containing the nanoparticle conjugate. The nanoparticle conjugate solution is put in the tube or vial, and subsequently dried to deposit the nanoparticle conjugate at the bottom, or on the inner wall.

In some embodiments, the device of the present invention comprises plastic wells containing the nanoparticle conjugate. The nanoparticle conjugate solution is put in the wells, and subsequently dried to deposit the nanoparticle conjugate at the bottom or on the inner wall. The completed device is dry without liquid component.

In one embodiment, the nanoparticle is conjugated to an antibody, and dried in the container. In another embodiment, the nanoparticle is conjugated to a binding domain of an antibody, including a single chain fragment variable (ScFv), or fragment antigen-binding region (Fab), or nanobody, and dried in the container.

In another embodiment, the nanoparticle is conjugated to an analyte binding protein, and dried in the container.

In another embodiment, the nanoparticle is conjugated to an aptamer, a DNA molecule, or an RNA molecule, and dried in the container.

In another embodiment, the nanoparticle is conjugated to an antigen, and dried in the container.

In another embodiment, the antigen itself is a conjugated molecule and is further conjugated to the nanoparticle and dried in the container.

The nanoparticles used in the conjugate of the present invention are particles ranging from 1 nm to 10,000 nm. In one embodiment, the nanoparticles are made from metal, inorganic or organic polymer materials. In one embodiment, the nanoparticles are selected from the group consisting of gold nanoparticles, silver nanoparticles, platinum nanoparticles, carbon nanoparticles, latex beads, magnetic nanoparticles, quantum dots, upconverting nanoparticles, and liposomes.

In one embodiment, the present invention is carried out in sandwich lateral flow immunoassay format, wherein the sample is added into the container containing the dried nanoparticle conjugate. Upon rehydration the nanoparticle conjugate is dissolved and contacted with the analytes if they are present in the sample. The sample is then applied on the sample pad of a lateral flow test strip containing a test line with a capturing reagent, and a control line containing another capturing reagent. The presence of analytes in the sample results in the retaining of the nanoparticle at the test line position that can be detected.

In another embodiment, the method of the present invention is carried out in a competitive lateral flow immunoassay format using dried nanoparticle conjugated antibody in the sample container. In this method, the sample is added into the container, and upon rehydration the antibody nanoparticles are dissolved and contacted with the analytes if they are present in the sample. The sample is then applied on the sample pad of a test strip containing a test line with the analyte molecule, and a control line containing a secondary capturing reagent. The presence of analytes in the sample results in the occupation of the antibody nanoparticle conjugate and the reduction or disappearance of the nanoparticle at the test line.

In another embodiment, the method of the present invention is carried out in a competitive lateral flow immunoassay format using dried nanoparticle conjugated antigen in the sample container. In this method, the sample is added into the container, and upon rehydration the antigen nanoparticles are dissolved and will compete with the analytes if they are present in the sample. The sample is then applied on the sample pad of a test strip containing a test line with analyte binding antibody, and a control line containing a secondary capturing reagent. The presence of analytes in the sample results in the occupation of the analyte binding antibody, and the reduction or disappearance of the nanoparticle at the test line.

In one embodiment, the presence of the analyte in the sample is detected by naked-eye inspection. In another embodiment, the presence of the analyte in the sample is detected by a reader that can measure optical signals such as absorbance, intensity, spectrum, fluorescence, or luminescence.

In another embodiment, the presence of the analyte in the sample is detected by a reader that can measure non-optical signals such as magnetic signals, thermal signals, or electrochemical signals.

In a specific embodiment, the present invention is used for detecting fentanyls in a sample. In one embodiment, the device comprises a sample vial and a test strip. The sample vial is prepared by first placing a gold nanoparticle conjugated anti-fentanyl antibody solution in the vial, then leaving the vial in a vented incubator until the solution is dried up. One advantage of this method is that the dried nanoparticle conjugate attaches firmly to the vial inner wall and will not detach during storage and transportation. The test strip comprises a sample pad, an analytic membrane, and an absorption pad. The analytic membrane is spotted with one test line and one control line. The test line is spotted with conjugated fentanyl molecules. Molecules that the fentanyl can be conjugated to include proteins and other large molecule weight molecules. The control line is spotted with an antibody binding reagent, such as a secondary antibody from a different species from that in which the anti-fentanyl antibody is derived, or protein G, or protein A, or protein L, or a mixture of any of the above. The device does not contain any liquid component, which is an advantage for production, storage, and transportation, and improves shelf life.

In one embodiment, the assay of the present invention is carried out by first adding the sample into the sample vial. Complete rehydration and mixture can be facilitated by pipetting, inverting, shaking or vortexing the sample vial. The vial can be capped during the mixing process. The test strip is then put in the vial and used as a dipstick. Full contact of the sample with the re-hydrated nanoparticle conjugate significantly increases the analyte detection sensitivity. This assay can detect <5 ng/ml fentanyl in a urine sample.

In another embodiment, the fentanyl detection device of the present invention further comprises a transfer pipet in addition to the sample vial and the lateral flow test strip. The assay using this device is carried out by first adding the sample into the sample vial. Complete rehydration and mixture can be facilitated by pipetting, inverting, shaking, or vortexing the sample vial. The vial can be capped during the mixing process. The sample is then transferred using the transfer pipet and applied to the sample zone of the lateral flow test strip.

In another embodiment, the fentanyl detection device of the present invention comprises a transfer pipet and the lateral flow test strip is sealed in a plastic cassette. The assay using this device is carried out by first adding the sample into the sample vial. Complete rehydration and mixture can be facilitated by pipetting, inverting, shaking, or vortexing the sample vial. The vial can be capped during the mixing process. The sample is then transferred using the transfer pipet and applied to the sample zone of the lateral flow test strip. This assay can detect <5 ng/ml fentanyl in a urine sample.

In one embodiment, the method and device of the present invention are used to detect fentanyl analogs in urine samples using a cassette format. This format can detect the following analogs with high sensitivity: para-chloroisobutyryl fentanyl, para-fluoro fentanyl, ocfentanil, sufentanil, butyryl fentanyl, cis-3-methylfentanyl, acryl fentanyl, carfentanil, benzodioxole fentanyl, crotonyl fentanyl, valeryl fentanyl, norcarfentanil, tetrahydrofuranyl fentanyl, despropionlyl 2′-fluoro ortho-fluorofentanyl, and furanyl fentanyl.

In another embodiment, the present invention relates to a kit using the method of the present invention using a dipstick format to detect the presence of fentanyl in urine samples. The conjugated nanoparticle is dried in a test tube. This kit is able to detect <1 ng/ml fentanyl in human urine samples.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Method and Devices to Increase the Sensitivity of Lateral Flow Assays

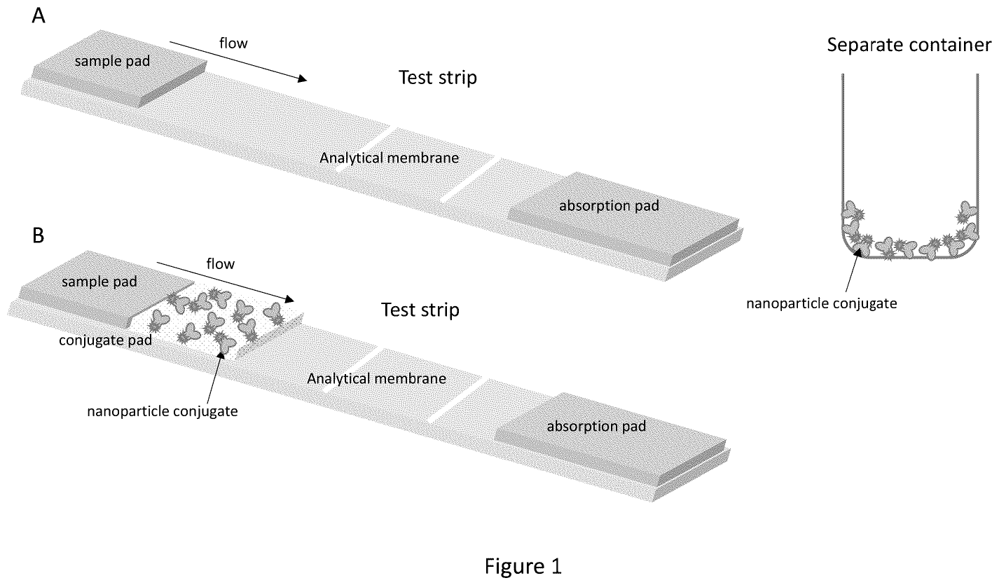

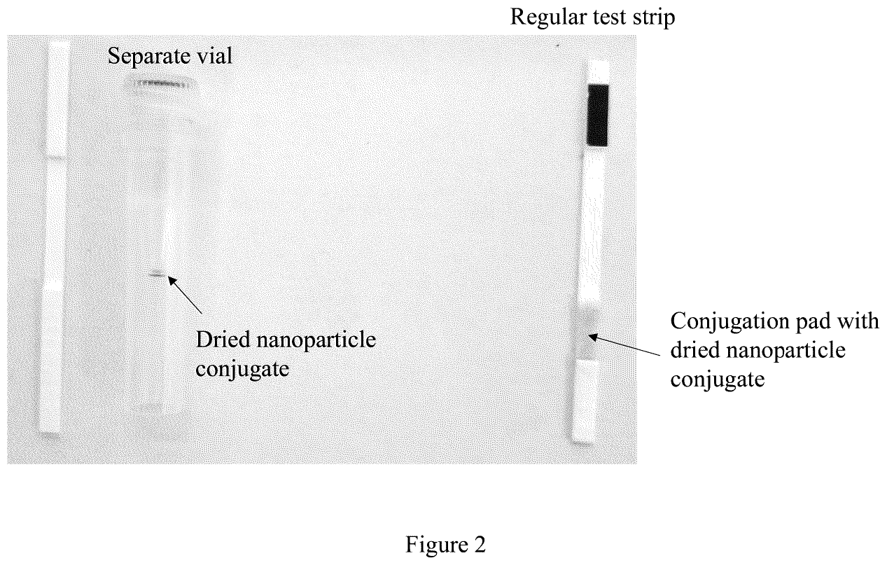

FIG. 1 shows a schematic view of the device of the present invention as compared to a conventional lateral flow device (A. the present invention; B. a conventional lateral flow device). FIG. 2 shows an example of the device of the present invention on the left, compared to the conventional lateral flow device on the right.

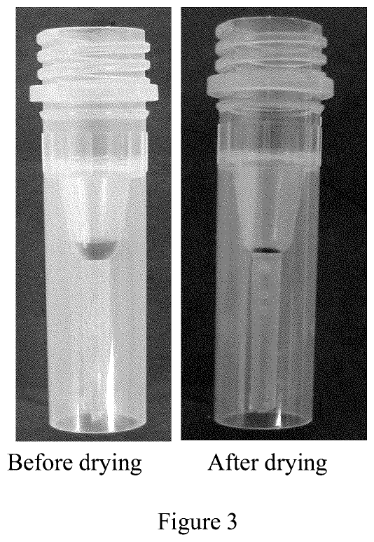

An example of the device of the present invention is shown in FIG. 3. The device comprises a tube or vial containing the nanoparticle conjugate. The nanoparticle conjugate solution is put in the tube or vial, and subsequently dried to deposit the nanoparticle conjugate at the bottom, or on the inner wall. The completed device is dry without liquid component.

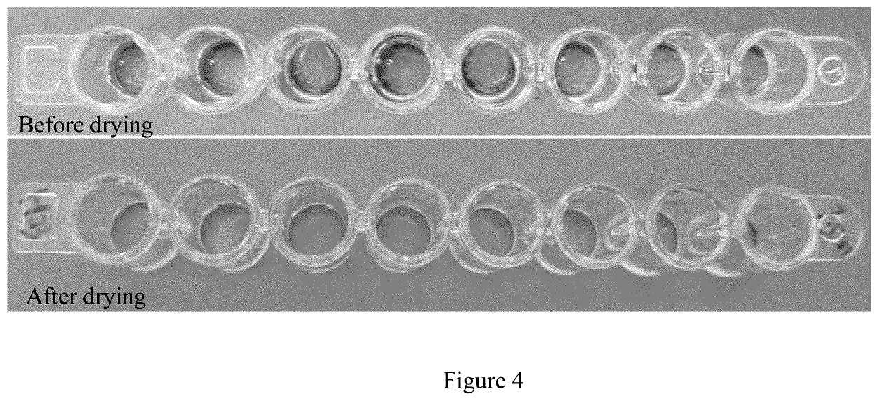

Another example of the device of the present invention is shown in FIG. 4. Here the device comprises plastic wells containing the nanoparticle conjugate. The nanoparticle conjugate solution is put in the wells, and subsequently dried to deposit the nanoparticle conjugate at the bottom or on the inner wall. The completed device is dry without liquid component. An example is shown in FIG. 4.

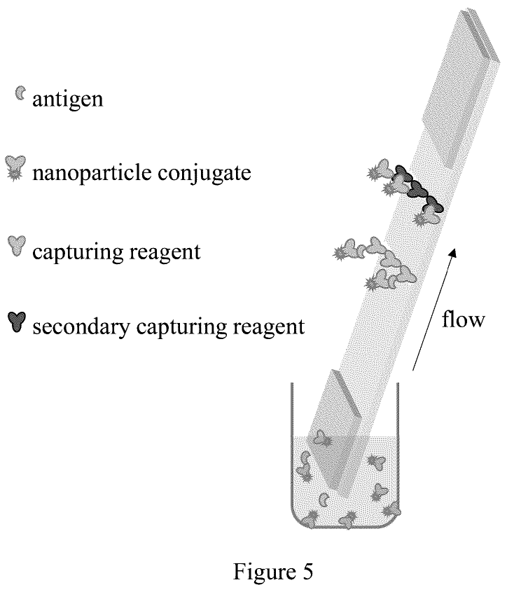

The method of the present invention can be carried out in sandwich lateral flow immunoassay format, as shown in FIG. 5. In this method, the sample is added into the container containing the dried nanoparticle conjugate. Upon rehydration the nanoparticle conjugate is dissolved and contacted with the analytes if they are present in the sample. The sample is then applied on the sample pad of a lateral flow test strip containing a test line with a capturing reagent, and a control line containing another capturing reagent. The presence of analytes in the sample results in the retaining of the nanoparticle at the test line position that can be detected, as depicted in FIG. 5.

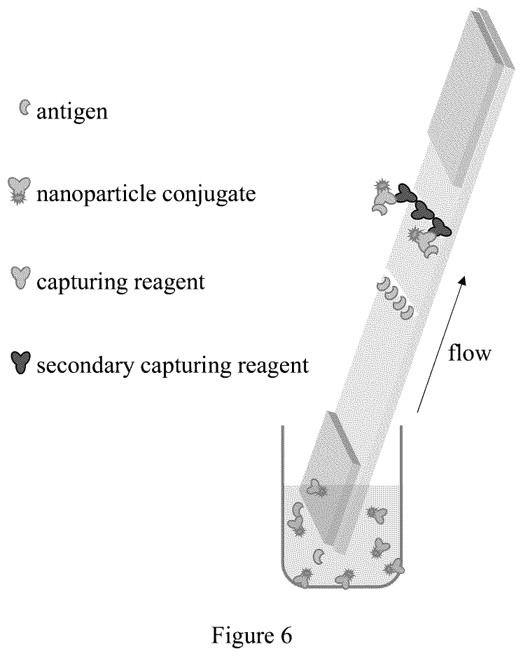

The method of the present invention can be carried out in competitive lateral flow immunoassay format using dried nanoparticle conjugated antibody in the sample container, as depicted in FIG. 6. In this method, the sample is added into the container. Upon rehydration the antibody nanoparticles are dissolved and contacted with the analytes if they are present in the sample. The sample is then applied on the sample pad of a test strip containing a test line with the analyte molecule, and a control line containing a secondary capturing reagent. The presence of analytes in the sample results in the occupation of the antibody nanoparticle conjugate, and the reduction or disappearance of the nanoparticle at the test line, as depicted in FIG. 6.

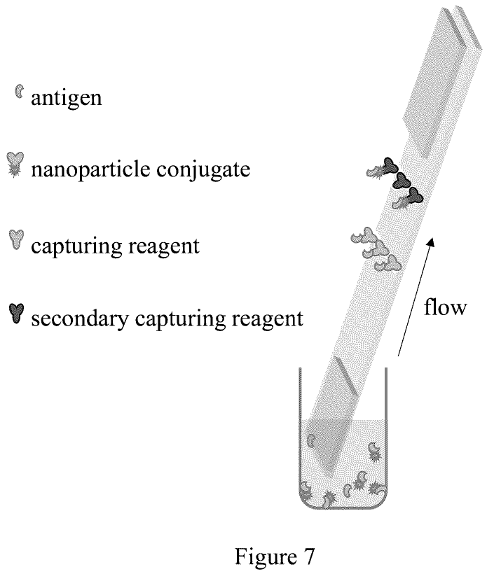

The method of the present invention can be carried out in competitive lateral flow immunoassay format using dried nanoparticle conjugated antigen in the sample container, as depicted in FIG. 7. In this method, the sample is added into the container and upon rehydration the antigen nanoparticles are dissolved that will compete with the analytes if they are present in the sample. The sample is then applied on the sample pad of a test strip containing a test line with analyte binding antibody, and a control line containing a secondary capturing reagent. The presence of analytes in the sample results in the occupation of the analyte binding antibody, and the reduction or disappearance of the nanoparticle at the test line, as depicted in FIG. 7.

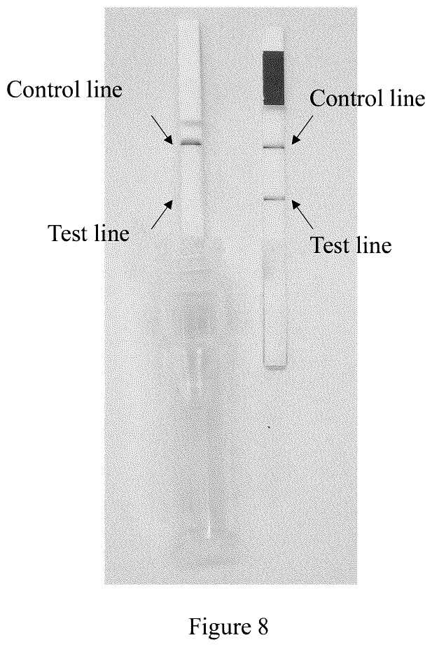

The assay of the present invention is carried out by first adding the sample into the sample vial. Complete rehydration and mixture can be facilitated by pipetting, inverting, shaking or vortexing the sample vial. The vial can be capped during the mixing process. The test strip is then put in the vial and used as a dipstick. Full contact of the sample with the re-hydrated nanoparticle conjugate significantly increases the analyte detection sensitivity. FIG. 8 left side shows that the assay of the present invention can detect at least 5 ng/ml fentanyl in a urine sample, leading to the complete disappearance of the test line. In contrast, regular lateral flow test strip failed to detect 50 ng/ml fentanyl in a urine sample, shown by the presence of the test line in FIG. 8, right side.

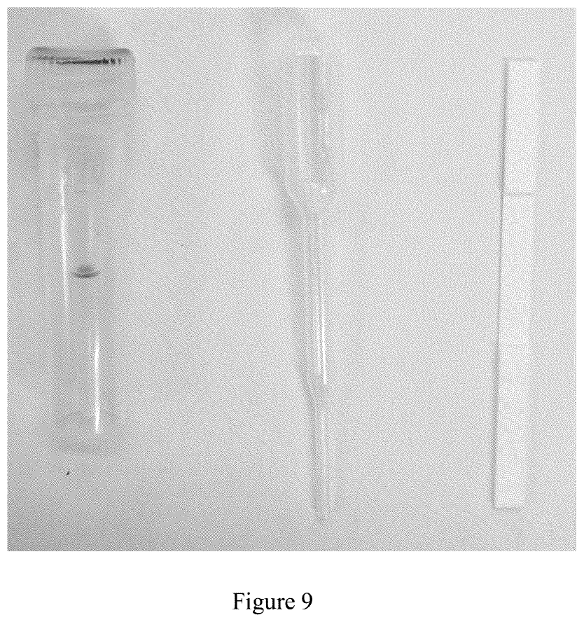

The fentanyl detection device of the present invention can further comprise a transfer pipet in addition to the sample vial and the lateral flow test strip, as depicted in FIG. 9. The assay using this device is carried out by first adding the sample into the sample vial. Complete rehydration and mixture can be facilitated by pipetting, inverting, shaking, or vortexing the sample vial. The vial can be capped during the mixing process. The sample is then transferred using the transfer pipet and applied to the sample zone of the lateral flow test strip.

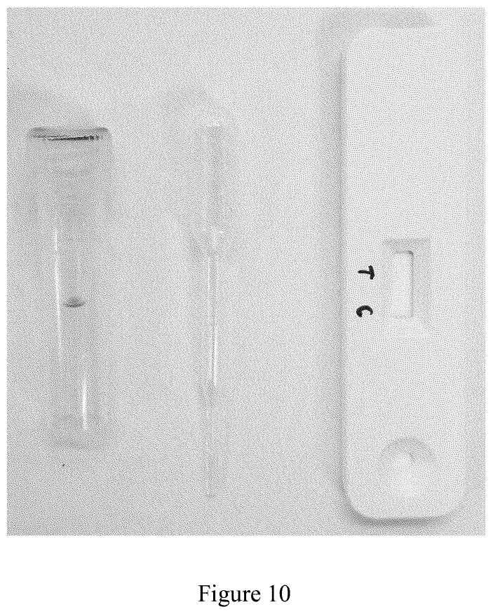

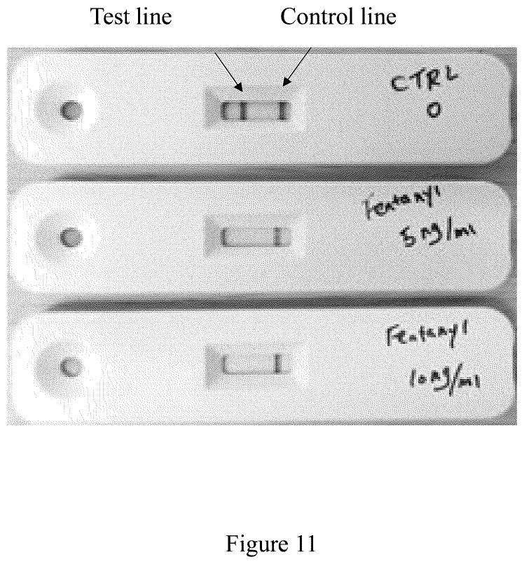

The fentanyl detection device of the present invention can further comprise a transfer pipet and the lateral flow test strip is sealed in a plastic cassette, as depicted in FIG. 10. The assay using this device is carried out by first adding the sample into the sample vial. Complete rehydration and mixture can be facilitated by pipetting, inverting, shaking, or vortexing the sample vial. The vial can be capped during the mixing process. The sample is then transferred using the transfer pipet and applied to the sample zone of the lateral flow test strip. FIG. 11 shows that high sensitivity is also achieved in this format, to detect at least 5 ng/ml fentanyl in a urine sample.

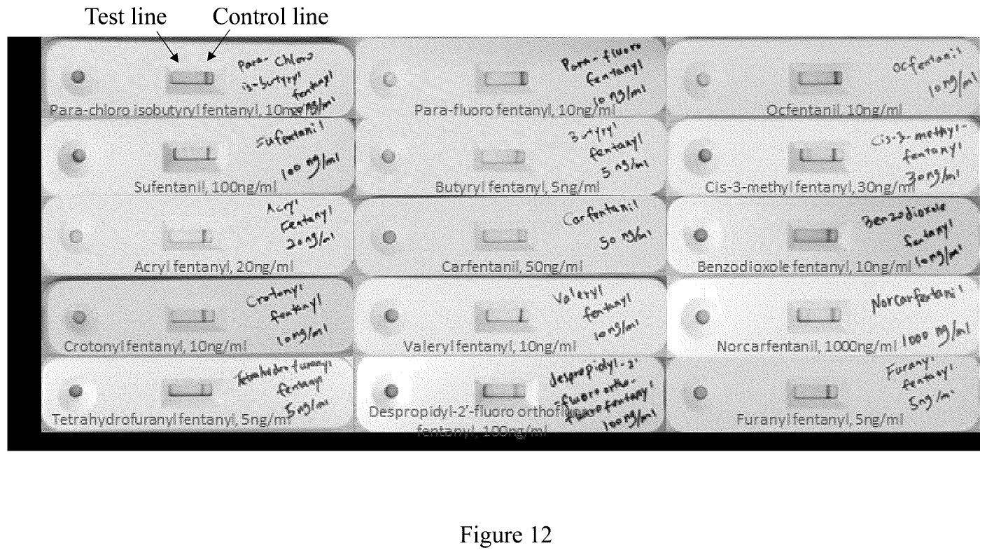

The method and device of the present invention can be used to detect fentanyl analogs in urine samples using a cassette format. FIG. 12 and Table 1 below depict results from an experiment showing that this format can detect at least 15 different analogs with high sensitivity. The tested compounds and the minimal sensitivity of the present device is listed below:

TABLE 1

| | Minimal |
|---|---|
| Compound | sensitivity |
| para-chloroisobutyryl fentanyl | 10 ng/ml |
| para-fluoro fentanyl | 10 ng/ml |
| Ocfentanil | 10 ng/ml |
| Sufentanil | 100 ng/ml |
| butyryl fentanyl | 5 ng/ml |
| cis-3-methylfentanyl | 30 ng/ml |
| acryl fentanyl | 20 ng/ml |
| Carfentanil | 50 ng/ml |
| benzodioxole fentanyl | 10 ng/ml |
| crotonyl fentanyl | 10 ng/ml |
| valeryl fentanyl | 10 ng/ml |
| Norcarfentanil | 1000 ng/ml |
| tetrahydrofuranyl fentanyl | 5 ng/ml |
| despropionlyl 2′-fluoro ortho- | 100 ng/ml |
| fluorofentanyl | |
| furanyl fentanyl | 5 ng/ml |

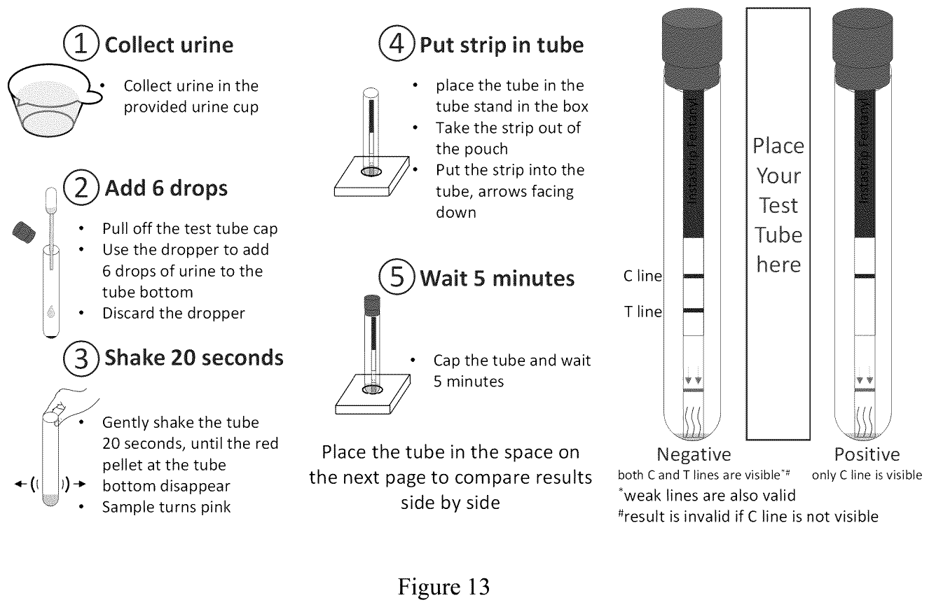

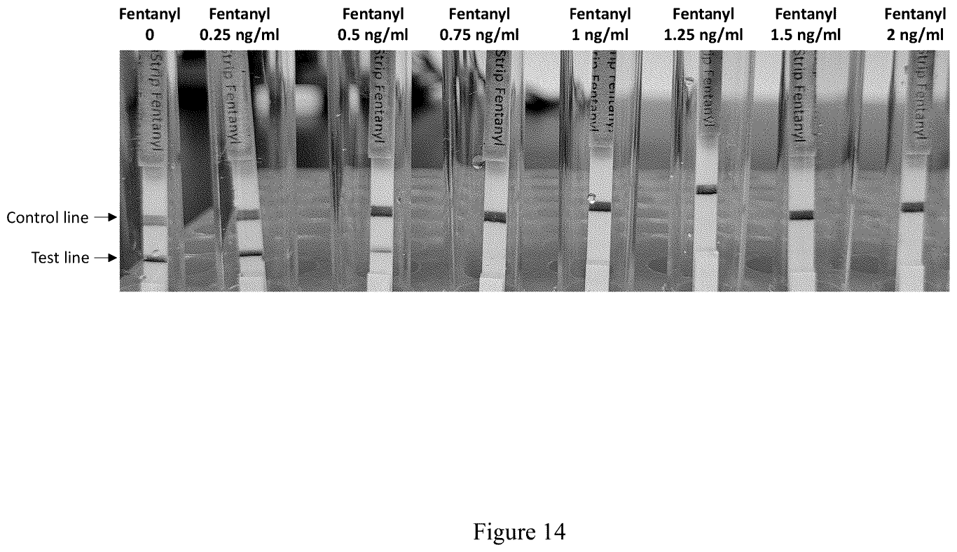

A kit was developed using the method of the present invention using a dipstick format to detect the presence of fentanyl in urine samples. The conjugated nanoparticle was dried in a test tube. The test procedure is illustrated in FIG. 13. With optimized antibodies and the quantities of the reagents, the kit was able to detect <1 ng/ml fentanyl in human urine samples, as shown in FIG. 14.

1. A method for detecting an analyte in a sample, comprising the steps of:
a) applying the sample to a container, wherein the container contains dried nanoparticle conjugate;
b) rehydrating the dried nanoparticle conjugate in the container, such that the analyte in the sample is in contact with the nanoparticle conjugate;
c) applying the sample to the sample zone of a lateral flow chromatographic test strip;
d) detecting the analyte on the detection zone of a lateral flow chromatographic test strip.

2. The method of claim 1, wherein the nanoparticle is selected from the group consisting of gold nanoparticles, silver nanoparticles, platinum nanoparticles, carbon nanoparticles, latex beads, magnetic nanoparticles, quantum dots, upconverting nanoparticles, and liposomes.

3. The method of claim 1, wherein the container is made of one or more materials selected from the group consisting of plastic, paper, metal, glass, and rubber.

4. The method of claim 1, further comprising the step of collecting the sample on a swab and then dissolving the sample in a solution.

5. The method of claim 1, wherein the lateral flow chromatographic test strip is put in the sample container and used as a dipstick.

6. The method of claim 1, wherein the sample is transferred from the container to the lateral flow chromatographic test strip using a transfer pipet.

7. The method of claim 6, wherein the lateral flow chromatographic test strip is in a cassette.

8. The method of claim 1, wherein the detection of the analyte is achieved by an immunoassay.

9. The method of claim 8, wherein the immunoassay uses an antibody selected from polyclonal antibodies, monoclonal antibodies, antibody fragments, or nanobodies.

10. The method of claim 1, wherein the detection of the analyte is achieved by a non-immunological assay.

11. The method of claim 10, wherein the detection of analyte uses analyte binding reagents selected from aptamers, DNA, RNAs, proteins, polymers, or small molecule compounds.

12. The method of claim 1, wherein the analyte is an antigen from a pathogen.

13. The method of claim 1, wherein the analyte is a disease marker.

14. The method of claim 1, wherein the analyte is a low molecular weight compound, including drugs of abuse.

15. The method of claim 1, wherein the analyte is a DNA molecule.

16. The method of claim 1, wherein the analyte is an RNA molecule.

17. The method of claim 1, wherein the lateral flow assay is carried out in sandwich immunoassay format.

18. The method of claim 1, wherein the lateral flow assay is carried out in competitive immunoassay format.

19. The method of claim 18, wherein the nanoparticle is conjugated to the analyte binding molecule.

20. The method of claim 18, wherein the nanoparticle is conjugated to the analyte.

21. The method of claim 18, wherein the nanoparticle is conjugated to a conjugated analyte.

22. The method of claim 1, wherein the lateral flow assay result is read by naked-eye detection.

23. The method of claim 1, wherein the lateral flow result is read by a strip reader.

24. The method of claim 23, wherein the lateral flow result is read by optical methods selected from absorbance reading, intensity reading, fluorescent reading, light spectrum reading, or luminescent reading.

25. The method of claim 23, wherein the lateral flow result is read by non-optical methods selected from magnetic reading, thermal reading, or electrochemical reading.

26. A device for detecting an analyte in a sample, comprising:
a) at least one container with dried nanoparticle conjugate, and/or
b) at least one lateral flow test strip, wherein the lateral flow matrix comprises at least one sample zone and at least one detection zone.

27. A kit for identifying fentanyl and analogs in a sample using the method according to claim 1.

28. A lateral flow cassette kit for identifying fentanyl and analogs in a sample using the device according to claim 26, comprising at least one container with dried antibody nanoparticle conjugate, and at least one lateral flow test strip cassette.

29. The kit of claim 28, wherein the sample is a fluid selected from blood, plasma, serum, urine, eye fluid, sweat, CSF, saliva or other body fluid.

30. The kit of claim 28, wherein the sample is a solution or buffer with or without the molecule to be detected.

31. The kit of claim 28, wherein the sample is a swab sample.

32. A lateral flow dipstick kit for identifying fentanyl and analogs in a sample using the device according to claim 26, comprising at least one container with dried antibody nanoparticle conjugate, and at least one lateral flow test strip dipstick.

33. The kit of claim 32, wherein the sample is a fluid selected from blood, plasma, serum, urine, eye fluid, sweat, CSF, saliva or other body fluid.

34. The kit of claim 32, wherein the sample is a solution or buffer with or without the molecule to be detected.

35. The kit of claim 32, wherein the sample is a swab sample.

36. The workflow of the kit of claim 32, which involves applying liquid samples to dissolve the dried gold nanoparticle in the tube, rehydrating the gold nanoparticles by shaking the tube, and then applying the test strip.

37. A method for reading lateral flow assay results as illustrated in FIG. 13, by placing the test strip on the side of a drawing, to compare with the negative and positive readouts.
